(19) United States
(12) Patent Application Publication
(10) Pub. No.: US 2024/0091493 A1
(43) Pub. Date: Mar. 21, 2024

(54) CATHETER HAVING REINFORCEMENT FIBERS AND RELATED DEVICES AND METHODS

(71) Applicant: Becton, Dickinson and Company, Franklin Lakes, NJ (US)

(72) Inventors: Jason R. Stats, Layton, UT (US); Daniel Blanchard, Bountiful, UT (US)

(21) Appl. No.: 18/238,980

(22) Filed: Aug. 28, 2023

Related U.S. Application Data
(60) Provisional application No. 63/407,460, filed on Sep. 16, 2022.

(51) Int. Cl. A61M 25/00; A61M 39/10

(52) U.S. Cl. CPC A61M 25/005; A61M 25/0012; A61M 25/0026; A61M 39/10; A61M 2025/0059; A61M 2039/1077; A61M 2205/0233

(57) ABSTRACT

A catheter assembly may include a catheter adapter, which may include a distal end and a proximal end. The catheter assembly may include a catheter extending from the distal end of the catheter adapter. The catheter may include a shaft and a lumen extending through the shaft. The shaft may include a wall and multiple reinforcement fibers within the wall. Each of the reinforcement fibers may have a greater tensile strength than the wall, facilitating kink-resistance and strength of the catheter.

### RELATED APPLICATIONS

This application claims the benefit of U.S. Provisional Patent Application No. 63/407,460, which was filed on Sep. 16, 2022, which is incorporated herein in its entirety.

### BACKGROUND

Catheters are commonly used for a variety of infusion therapies. For example, catheters may be used for infusing therapeutic agents or fluids into a patient. Catheters may also be used for withdrawing blood from the patient. There are a variety of catheters commonly used in a medical setting, including, for example, peripherally-inserted central catheters, midline catheters, central venous catheters, dialysis catheters, and arterial catheters. In some instances, catheters may be inserted into a blood vessel of a patient via the modified Seldinger technique or the Seldinger technique. A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter.

The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. In order to verify proper placement of the introducer needle and/or the catheter in the blood vessel, a clinician generally confirms that there is “flashback” of blood in a flashback chamber of the catheter assembly. Once placement of the needle has been confirmed, the catheter may be left in place for future blood withdrawal or fluid infusion.

Unfortunately, kinks may form in a shaft of a catheter during use when the shaft is bent beyond its minimum bend radius or kink radius. When a kink forms in the shaft of the catheter during use, one or more internal lumens within the shaft can become obstructed, and the flow of a therapeutic agent or fluid through the catheter can be interrupted. It is therefore desirable to provide a device that resists or prevents such kink-related obstructions and interruptions in therapeutic agent or fluid delivery.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter assembly, as well as related devices and methods. In some embodiments, the catheter assembly may include a catheter adapter, which may include a distal end and a proximal end. In some embodiments, the catheter assembly may include a catheter extending from the distal end of the catheter adapter. In some embodiments, the catheter may include a shaft and a lumen extending through the shaft. In some embodiments, the shaft may include a wall and multiple reinforcement fibers within the wall. In some embodiments, each of the reinforcement fibers may have a greater tensile strength than the wall, which may facilitate kink-resistance and strength of the catheter. In some embodiments, each of the reinforcement fibers may have a greater durometer than the wall, which may facilitate kink-resistance and strength of the catheter. In some embodiments, the reinforcement fibers may improve elastic elongation of the catheter at a particular durometer or tensile strength of the catheter itself. In some embodiments, the reinforcement fibers may have a decreased elastic elongation compared to the wall.

In some embodiments, the reinforcement fibers may include nylon, aromatic polyamide fiber, KEVLAR™, carbon fiber, carbon nanotubes, fiber glass, silver nanowires, polymer fibers, or another suitable fiber material. In some embodiments, a length of each of the reinforcement fibers may be between 10 microns and 1,000 microns. In some embodiments, the length of the reinforcement fibers may be short enough to facilitate bending of the catheter and allow the catheter to maintain flexibility, while the reinforcement fibers also provide strength and anti-kink properties to the catheter. In some embodiments, the reinforcement fibers can also enhance other properties of the catheter, including electrical conductivity, stiffness, and/or thermal conductivity. In some embodiments, each of the reinforcement fibers may be encapsulated by the wall. In some embodiments, a distance of one or more of the reinforcement fibers from a longitudinal axis of the catheter may be different such that depths of the reinforcement fibers within the shaft may vary. In some embodiments, a distal end of one or more of the reinforcement fibers may overlap with a proximal end of one or more other of the reinforcement fibers, which may provide strength to the catheter. In some embodiments, the reinforcement fibers may be oriented generally parallel to a central longitudinal axis of the catheter, which may increase stiffness and strength of the catheter and resist kinking.

Kink-resistance is of high importance for a catheter to maintain an open flow path. However, because it is often desired to have a catheter lumen as large as possible for the best flow rates while at a same time having a small catheter outer diameter, it has become increasingly difficult to provide kink-resistance with just catheter lumen shapes and wall thicknesses. Catheter kink can be hard to avoid due to the competing objectives of small catheter outer diameter and high flow rates through the catheter. In some embodiments, the reinforcement fibers may include relatively short strains of fiber that allow the shaft and the wall to be thin, facilitating high flow rates, and that also allow the shaft to bend without kinking so easily. In some embodiments, the catheter may include a peripheral intravenous catheter (PIVC), a peripherally-inserted central catheter (PIVC), dialysis catheter, a midline catheter, a central venous catheter (CVC), or another suitable catheter.

In some embodiments, the shaft may include the wall, a stripe or an annular layer within the wall, and the reinforcement fibers within the stripe or the annular layer. In some embodiments, the stripe or the annular layer may be conductive, which may provide a pathway for sending/receiving an electrical signal through the catheter. In some embodiments, each of the reinforcement fibers may have a greater tensile strength than the wall. In some embodiments, each of the reinforcement fibers have a greater tensile strength than the stripe or the annular layer. In some embodiments, the wall may include an inner surface forming the lumen and an outer surface forming an exterior of the catheter.

In some embodiments, the stripe may be proximate the inner surface and extend partially through the wall. In some embodiments, the stripe may be proximate the outer surface and extend partially through the wall. In some embodiments, the stripe may extend through the wall from the inner surface to the outer surface. In some embodiments, the shaft may include multiple stripes, which may include the stripe. In some embodiments, the stripes may be spaced around a circumference of the shaft. In some embodiments, the wall may completely surround each of the stripes. In some embodiments, the catheter may include another lumen extending through the shaft. In some embodiments, the shaft may include the stripe and another stripe. In some embodiments, wherein the stripe and the other stripe may be opposite each other between the lumen and the other lumen and completely surrounded by the wall.

In some embodiments, the annular layer may include an inner surface forming the lumen and an outer surface proximate the wall. In some embodiments, the annular layer may extend along all or a portion of an entire length of the catheter. In some embodiments, the shaft may include the annular layer within the wall, and the wall may sandwich the annular layer.

In some embodiments, the wall may be constructed of a first material, which may include a resin, such as polyurethane, for example. In some embodiments, the resin may provide some kink resistance. In some embodiments, the first material may include a polyurethane product such as VIALON™ biomaterial available from Becton, Dickinson and Company of Franklin Lakes, New Jersey. In some embodiments, the first material may be reinforced with the reinforcement fibers to provide additional kink-resistance. In some embodiments, the stripe or the annular layer may be constructed of a second material different from the first material. In some embodiments, the second material may be in a same class of materials as the first material to facilitate bonding between the first material and the second material during extrusion. In some embodiments, the first material and/or the second material may include at least one of polyurethane, nylon, polyether block amide (PEBA), silicone, polypropylene, polyethylene, or the like.

In some embodiments, one or more of the following may be radiopaque (may include barium sulfate or another suitable radiopaque compound) to facilitate visualization of the catheter during a medical procedure: the reinforcement fibers, the stripe, and the annular wall. In some embodiments, one or more of the following may be biocompatible, allowing insertion into a blood vessel of a patient: the reinforcement fibers, the wall, the stripe, and the annular layer. In some embodiments, the first material or material of the wall of the catheter may include a conductive additive, which may provide a pathway for sending/receiving an electrical signal through the catheter. In some embodiments, the reinforcement fibers may include one or more antimicrobial, anticoagulatory, or anti-fouling compounds.

In some embodiments, a method of manufacturing the catheter may include providing an extrusion pin within an extrusion die. In some embodiments, the extrusion pin may include a body having a first diameter that tapers down to a nose having a second diameter. In some embodiments, the method may include flowing a polymeric material around the exterior surface of the nose and through the extrusion pin. In some embodiments, the polymeric material may include the reinforcement fibers. In some embodiments, in response to the polymeric material flowing around the exterior surface of the nose and through the extrusion pin, the reinforcement fibers may be configured to orient towards a position aligned with the central longitudinal axis of the catheter. In some embodiments, the method may include drawing down and/or pulling the polymeric material out of the extrusion die. In some embodiments, in response to drawing down and/or pulling the polymeric material out of the extrusion die, the reinforcement fibers may be configured to orient further towards the position aligned with the central longitudinal axis of the catheter.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system, according to some embodiments;

FIG. 1B is a longitudinal cross-sectional view of an example catheter, illustrating example reinforcement fibers in an unaligned or random orientation, according to some embodiments;

FIG. 2A is a longitudinal cross-sectional view of an example extrusion system, according to some embodiments;

FIG. 2B is an upper perspective view of an example extrusion pin, according to some embodiments;

FIG. 2C is a longitudinal cross-sectional view of an example catheter, illustrating example reinforcement fibers in an orientation generally aligned or parallel to a longitudinal axis of the catheter, according to some embodiments;

FIG. 3 is a longitudinal cross-sectional view of an example catheter, illustrating example reinforcement fibers that are overlapping and in the orientation generally aligned or parallel to the longitudinal axis of the catheter, according to some embodiments;

FIG. 4A is a transverse cross-sectional view of an example catheter, illustrating multiple example stripes, according to some embodiments;

FIG. 4B is a longitudinal cross-sectional view of the catheter of FIG. 4A, illustrating the stripes, according to some embodiments;

FIG. 5A is a transverse cross-sectional view of an example catheter, illustrating an example stripe, according to some embodiments;

FIG. 5B is a longitudinal cross-sectional view of the catheter of FIG. 5A, illustrating the stripe, according to some embodiments;

FIG. 6A is a transverse cross-sectional view of an example catheter, illustrating multiple example stripes, according to some embodiments;

FIG. 6B is a longitudinal cross-sectional view of the catheter of FIG. 6A, illustrating the stripes, according to some embodiments;

FIG. 7A is a transverse cross-sectional view of an example catheter, illustrating an example annular layer, according to some embodiments;

FIG. 7B is a longitudinal cross-sectional view of the catheter of FIG. 7A, illustrating the annular layer, according to some embodiments;

FIG. 8A is a transverse cross-sectional view of an example catheter, illustrating the annular layer, according to some embodiments;

FIG. 8B is a longitudinal cross-sectional view of the catheter of FIG. 8A, illustrating the annular layer, according to some embodiments;

FIG. 9A is a transverse cross-sectional view of an example catheter, illustrating the annular layer, according to some embodiments;

FIG. 9B is a longitudinal cross-sectional view of the catheter of FIG. 9A, illustrating the annular layer, according to some embodiments;

FIG. 10 is a transverse cross-sectional view of an example catheter, illustrating multiple example lumens and example stripes, according to some embodiments; and

FIG. 11 is a transverse cross-sectional view of an example catheter, illustrating the annular layer, according to some embodiments.

### DESCRIPTION OF EMBODIMENTS

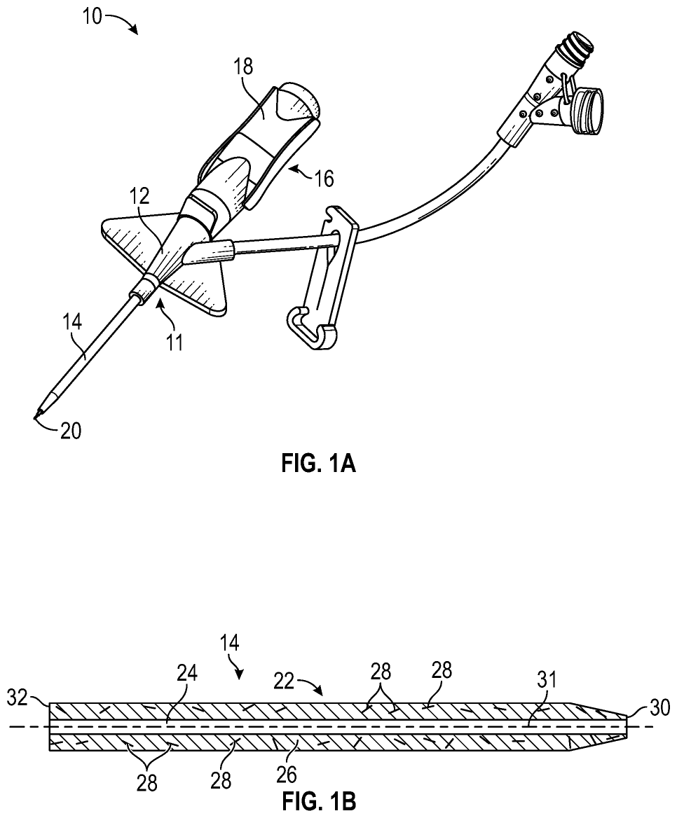

Referring now to FIG. 1A, in some embodiments, a catheter system 10 may include a catheter assembly 11, which may include a catheter adapter 12 and a catheter 14 extending distally from the catheter adapter 12. In some embodiments, the catheter system 10 may include a needle assembly 16, which may include a needle hub 18 and an introducer needle 20. In some embodiments, the catheter 14 may include be over-the-needle, and the introducer needle 20 may extend through the catheter 14 when the catheter system 10 is in an insertion configuration ready for insertion into a patient, as illustrated, for example in FIG. 1A. The catheter system 10 is just one example of the various catheter systems that may be used according to the present disclosure. In some embodiments, the catheter assembly 11 may include any suitable catheter assembly or needle assembly. In some embodiments, a catheter according to the present disclosure may not be over-the-needle and may include any other suitable type of catheter. In some embodiments, the catheter described according to the present disclosure may include a peripherally-inserted central catheter, a midline catheter, a central venous catheter, a dialysis catheter, an arterial catheter, or another type of catheter. In some instances, a catheter according to the present disclosure may be inserted into the blood vessel of a patient via the modified Seldinger technique, the Seldinger technique, or another suitable technique.

Referring now to FIG. 1B, in some embodiments, the catheter 14 may include a shaft 22 and a lumen 24 extending through the shaft 22. In some embodiments, the shaft 22 may include a wall 26 and multiple reinforcement fibers 28 within the wall 26. In some embodiments, the wall 26 may be annular. In some embodiments, the catheter 14 may include a distal end 30, which may be tapered, and a proximal end 32. In some embodiments, each of the reinforcement fibers 28 may include a greater tensile strength than the wall 26, which may facilitate kink-resistance and strength of the catheter 14. In some embodiments, each of the reinforcement fibers 28 may be encapsulated by the wall 26. In some embodiments, the catheter 14 may include a central longitudinal axis 31.

In some embodiments, the reinforcement fibers 28 may include nylon, aromatic polyamide fiber, polyurethane, KEVLAR™, carbon fiber, carbon nanotubes, fiber glass, polymer fibers, silver nanowires, or another suitable fiber material. In some embodiments, a length of each of the reinforcement fibers 28 may be between 10 microns and 1,000 microns. In some embodiments, the length of the reinforcement fibers 28 may be between 10 microns and 100 microns, 100 microns and 200 microns, 200 microns and 300 microns, 300 microns and 400 microns, 400 microns and 500 microns, 500 microns and 600 microns, 600 microns and 700 microns, 700 microns and 800 microns, 800 microns and 900 microns, or 900 microns and 1,000 microns. In some embodiments, the length of the reinforcement fibers 28 may be short enough to facilitate bending of the catheter 14 and allow the catheter 14 to maintain flexibility, while the reinforcement fibers 28 also provide strength and anti-kink properties to the catheter 14. In some embodiments, each of the reinforcement fibers 28 may be uniform in length to facilitate predictable anti-kink properties of the catheter 14.

In some embodiments, the wall 26 may be constructed of a polymeric material, which may include a resin, such as polyurethane, for example. In some embodiments, the resin may provide some kink resistance. In some embodiments, the polymeric material may include a polyurethane product such as VIALON™ biomaterial available from Becton, Dickinson and Company of Franklin Lakes, New Jersey.

In some embodiments, an extrusion system used to create a particular catheter 14 of the present disclosure may vary. For example, the extrusion system may include multiple extrusion dies, multiple extrusion pins, and/or one or more baffles. In some embodiments, the catheter 14 extruded with the reinforcement fibers 28 may be created using any number of standard extrusion processes known in the art, which may be specific for a particular thermoset or a particular thermoplastic. In some embodiments, the extrusion process may include forcing the polymeric material through an extrusion die or dies and drawing the extrusion down, which may cause the reinforcement fibers 28 to orient generally in an axial direction of the extrusion or along a central longitudinal axis of the catheter 14.

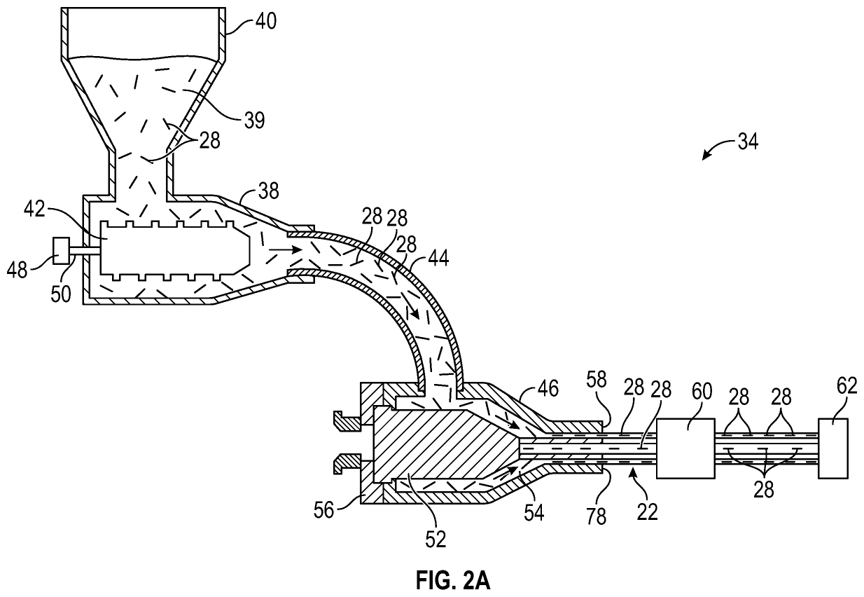

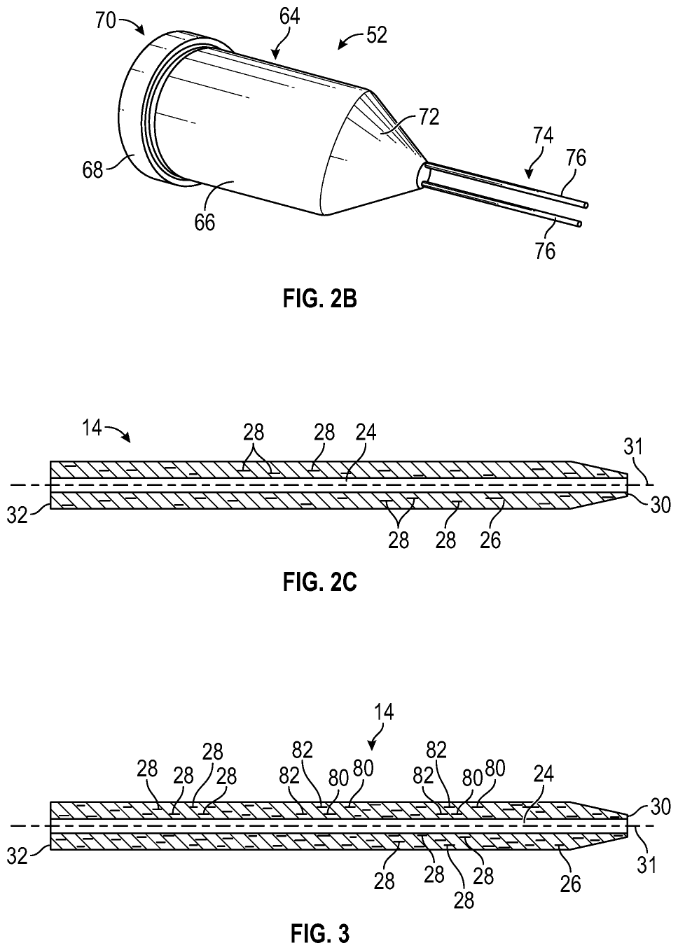

Referring now to FIG. 2A, an extrusion system 34 is illustrated, according to some embodiments. The extrusion system 34 is just one example of an extrusion system. In some embodiments, the extrusion system 34 may vary from what is illustrated in FIG. 2A. In some embodiments, the extrusion system 34 may be used to orient the reinforcement fibers 28 parallel or generally parallel to a central longitudinal axis 31 of the catheter 14, which may increase stiffness and strength of the catheter 14 and resist kinking. In some embodiments, the extrusion system 36 may include an extruder 38 which may be used to form the shaft 22 into a tubular shape. In some embodiments, the extruder 38 may include a hopper 40 containing a quantity of the reinforcement fibers 28 mixed with the polymeric material 39 for forming the tubular shape. In some embodiments, a screw 42 may deliver the polymeric material 39 and the reinforcement fibers 28 through a conduit 44 to an extrusion die 46. For example, a motor 48 may delivery rotational power through a shaft 50 to the screw 42, pushing the polymeric material 39 and the reinforcement fibers 28 toward the extrusion die 46 through the conduit 44.

In some embodiments, a mandrel or extrusion pin 52 may be located within the extrusion die 46. In some embodiments, the extrusion pin 52 may be heated to an elevated temperature during the extrusion process. In some embodiments, the extrusion pin 52 may be housed within a cavity 54 of the extrusion die 46, leaving a gap between the extrusion pin 52 and the extrusion die 46 for molten polymeric material 39 and the reinforcement fibers 28 to flow (as shown by arrows in FIG. 2A). In some embodiments, the extrusion pin 52 may be positioned in the extrusion die 46 such that a central longitudinal axis of the extrusion pin 52 is longitudinally aligned with a central longitudinal axis of the extrusion die 46. In some embodiments, the extrusion pin 52 may be secured in the extrusion die 46 by securing an end cap 56 onto a rear of the extrusion die 46 with one or more fasteners or the like.

In some embodiments, after the shaft 22 that is extruded exits the extrusion die 46 through an opening 58 of the extrusion die 46, the shaft 22 may pass through a water bath 60 or other cooling apparatus. In some embodiments, the water bath 60 may help cool the shaft 22 by extracting heat energy from the shaft 22 into water or other fluid by conduction. In some embodiments, the extrusion system 34 may also include a puller 62 that controls a pull rate (i.e., longitudinal rate of advancement) of the shaft 22 out of the extrusion die 46.

Referring now to FIG. 2B, the extrusion pin 52 is illustrated, according to some embodiments. In some embodiments, the extrusion pin 52 may include a body 64 including a generally cylindrical portion 66 and/or a base 68 at a rear end 70 of the body 64 of the extrusion pin 52. In some embodiments, the extrusion pin 52 may also include a conical portion 72 forward of the cylindrical portion 66 and taper downward a forward end of the body 64. In some embodiments, the conical portion 72 of the body 64 may taper to a nose 74. In some embodiments, the body 64 may have a first diameter that tapers down to the nose 74 having a second diameter. In some embodiments, the nose 74 may include one or more generally cylindrical portions 76, depending on, for example, a desired number of lumens within the catheter 14. FIG. 2B illustrates two generally cylindrical portions for the formation of two lumens within the catheter 14, according to some embodiments. Referring now to FIGS. 2A-2B, in some embodiments, the nose 74 may extend to a forward end 78 of the extrusion pin 52. A shape of the nose 74 and/or the extrusion pin 52 may vary according to, for example, a desired shape or composition of a particular catheter, as is known in the art.

In some embodiments, the polymeric material 39 and the reinforcement fibers 28 may flow around the exterior surface of the nose 74 and through the extrusion pin 52. In some embodiments, in response to the polymeric material 39, mixed with the reinforcement fibers 28, flowing around the exterior surface of the nose 74 and through the extrusion pin 52, the reinforcement fibers 28 may be configured to orient towards a position aligned with the central longitudinal axis 31 of the catheter 14. In some embodiments, the polymeric material 39 and the reinforcement fibers 28 out of the extrusion die 46. In some embodiments, in response to drawing down and/or pulling the polymeric material 39, mixed with the reinforcement fibers 28, out of the extrusion die 46, the reinforcement fibers 28 may be configured to orient further towards the position aligned with the central longitudinal axis 31 of the catheter 14.

Referring now to FIGS. 2C-3, the catheter 14 having the reinforcement fibers 28 generally aligned with the central longitudinal axis 31 of the catheter 14 is illustrated, according to some embodiments. In some embodiments, the reinforcement fibers 28 may be oriented generally parallel to the central longitudinal axis 31 of the catheter 14, which may increase stiffness and strength of the catheter 14 and resist kinking. In some embodiments, a distance of one or more of the reinforcement fibers 28 from the central longitudinal axis 31 of the catheter 14 may be different such that depths of the reinforcement fibers 28 within the shaft 22 may vary. As illustrated in FIG. 3, in some embodiments, a distal end 80 of one or more of the reinforcement fibers 28 may overlap with a proximal end 82 of one or more other of the reinforcement fibers 28, which may provide strength to the catheter 14.

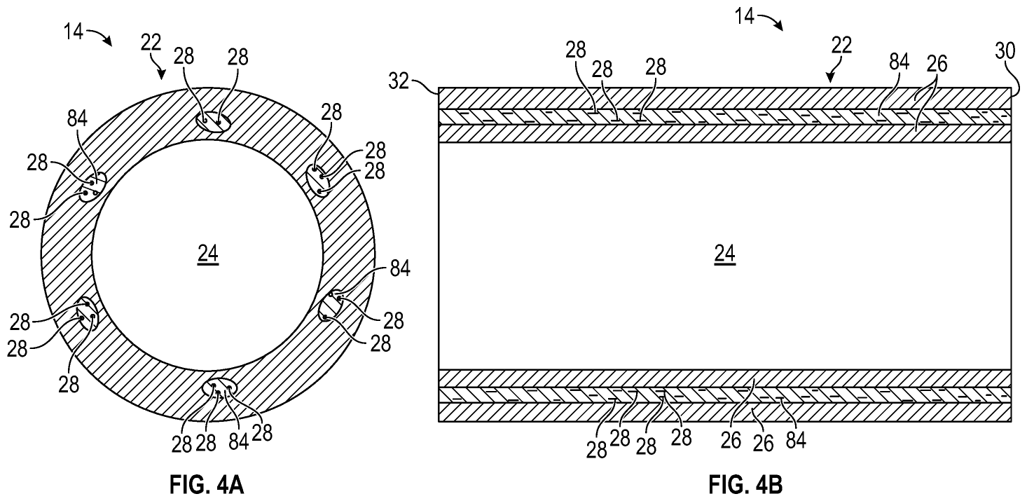

Referring now to FIGS. 4-6, in some embodiments, the shaft 22 may include the wall 26, a stripe 84 within the wall 26, and the reinforcement fibers within the stripe 84. In some embodiments, the stripe 84 may be conductive, which may provide a pathway for sending/receiving an electrical signal through the catheter 14. In some embodiments, the stripe 84 may have electrical conductivity and/or thermal conductivity. In some embodiments, each of the reinforcement fibers 28 may have a greater tensile strength than the wall 26. In some embodiments, each of the reinforcement fibers 28 have a greater tensile strength than the stripe 84. In some embodiments, the reinforcement fibers 28 may improve an overall tensile strength of the catheter 14.

As illustrated in FIGS. 4A-4B, in some embodiments, multiple stripes 84 may be spaced around a circumference of the shaft 22. In some embodiments, the wall 26 may completely surround each of the stripes 84. In some embodiments, the stripes 84 may extend along all or a portion of an entire length of the catheter 14.

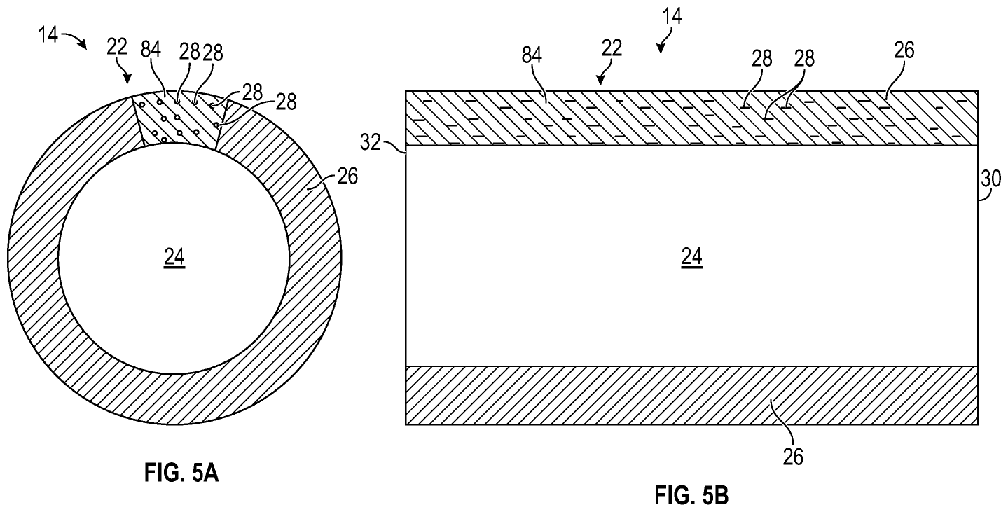

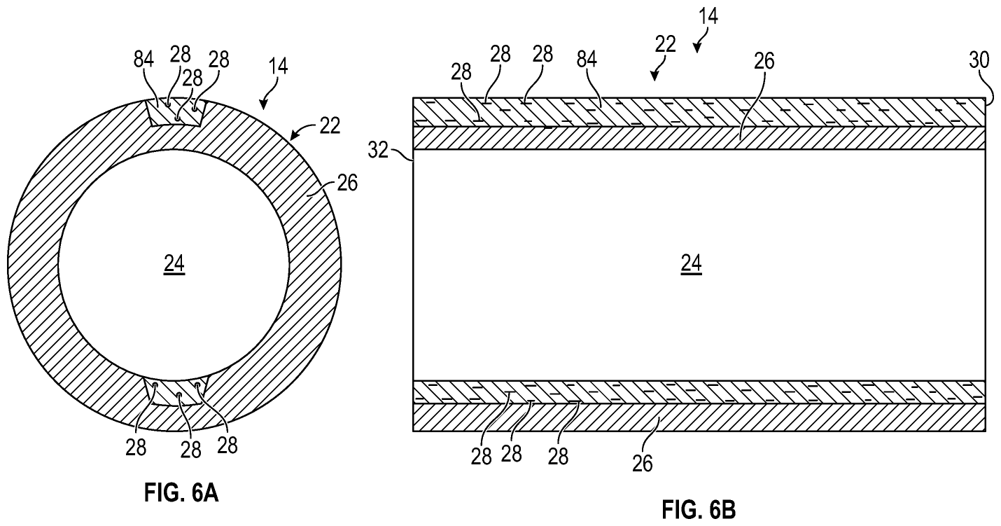

In some embodiments, the wall 26 may include an inner surface forming the lumen 24 and an outer surface forming an exterior and outer diameter of the catheter 14. As illustrated in FIG. 5A-5B, in some embodiments, the stripe 84 may extend through the wall 26 from the inner surface to the outer surface. As illustrated in FIGS. 6A-6B, in some embodiments, the stripe 84 may be proximate the inner surface and extend partially through the wall 26. As also illustrated in FIGS. 6A-6B, in some embodiments, the stripe 84 may be proximate the outer surface and extend partially through the wall 26. As further illustrated in FIGS. 6A-6B, in some embodiments, the shaft may include multiple stripes 84.

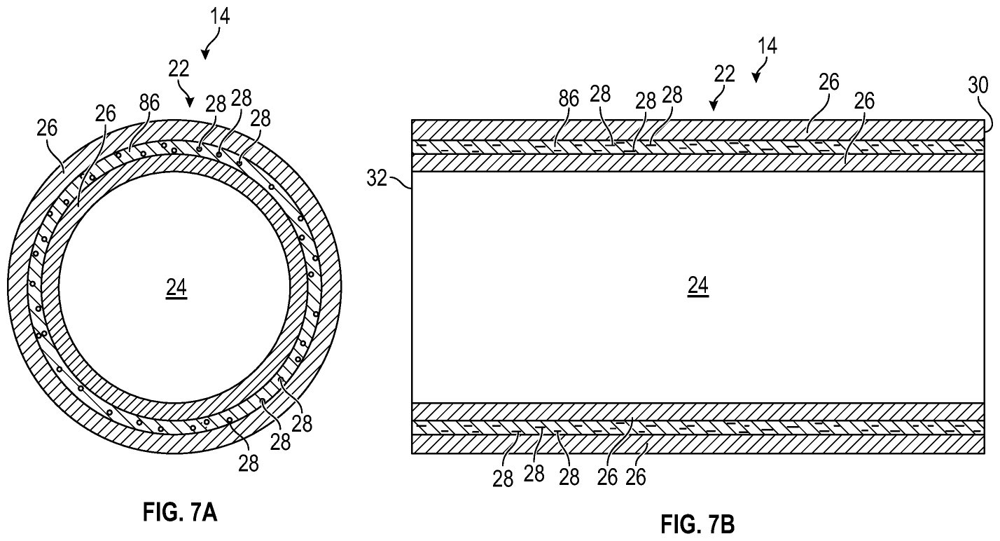

Referring now to FIGS. 7-9 and 11, in some embodiments, the shaft 22 may include the wall 26, an annular layer 86 within the wall 26, and the reinforcement fibers 28 within the annular layer 86. In some embodiments, the annular layer 86 may be conductive, which may provide a pathway for sending/receiving an electrical signal through the catheter 14. In some embodiments, the annular layer 86 may have electrical conductivity and/or thermal conductivity. In some embodiments, each of the reinforcement fibers 28 may have a greater tensile strength than the wall 26. In some embodiments, each of the reinforcement fibers 28 have a greater tensile strength than the annular layer 86.

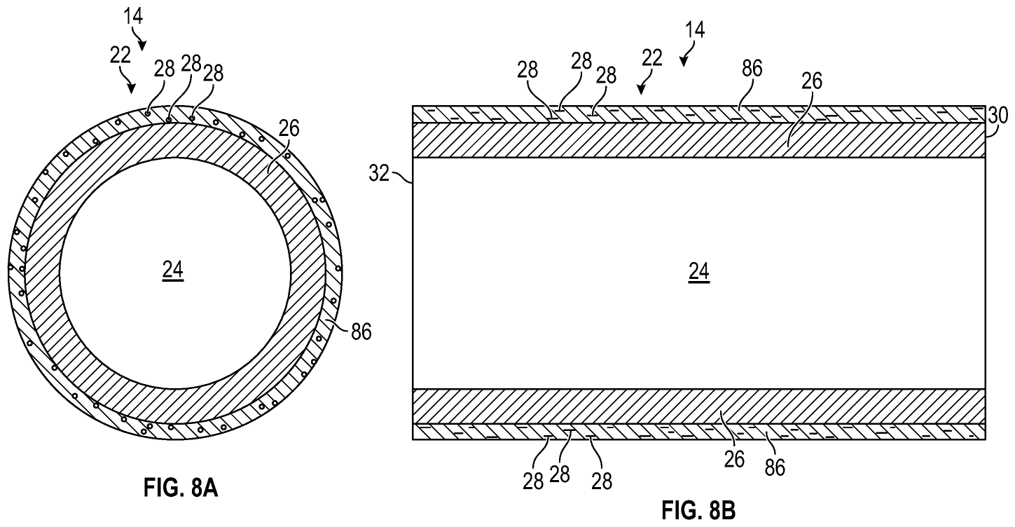

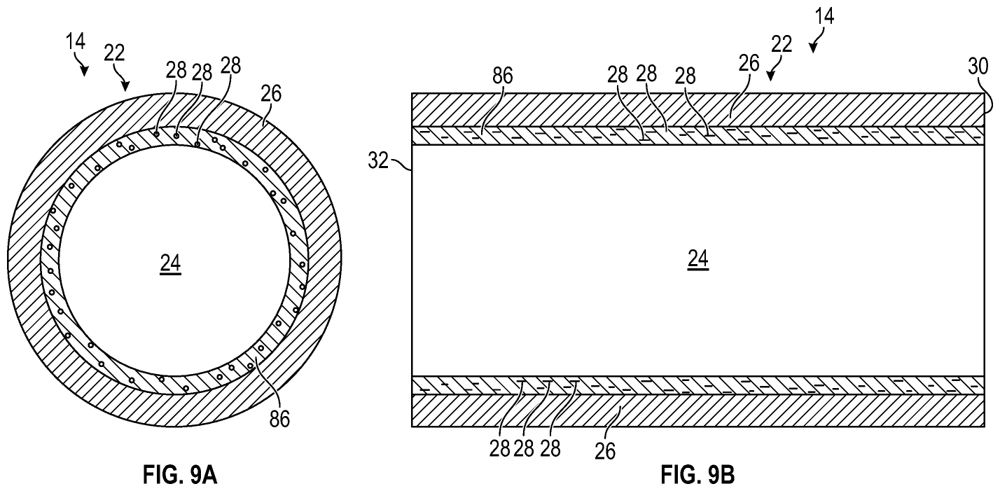

In some embodiments, the shaft 22 may include the annular layer 86 within the wall 26, and the wall 26 may sandwich the annular layer 86, as illustrated in FIGS. 7A-7B. As illustrated in FIGS. 8A-8B, in some embodiments, the annular layer 86 may include an inner surface proximate the wall 26 and an outer surface forming an exterior and outer diameter of the catheter 14. As illustrated in FIGS. 9A-9B, in some embodiments, the annular layer 86 may include an inner surface forming the lumen 24 and an outer surface proximate the wall 26. In some embodiments, the annular layer 86 may extend along all or a portion of the entire length of the catheter 14.

In some embodiments, the wall 26 may be constructed of a first material, such as the polymeric material 39 (see, for example, FIG. 2A). In some embodiments, the first material may include a resin, such as polyurethane, for example. In some embodiments, the resin may provide some kink resistance. In some embodiments, the first material may include a polyurethane product such as VIALON™ biomaterial available from Becton, Dickinson and Company of Franklin Lakes, New Jersey. In some embodiments, the first material may be reinforced with the reinforcement fibers 28 to provide additional kink-resistance.

Referring now to FIGS. 4-11, in some embodiments, the stripe 84 or the annular layer 86 may be constructed of a second material, which may be in a same class of materials as the first material to facilitate bonding between the first material and the second material during extrusion. In some embodiments, one or more of the following may be radiopaque (may include barium sulfate or another suitable radiopaque compound) to facilitate visualization of the catheter 14 during a medical procedure: the reinforcement fibers 28, the stripe 84, and the wall 26. In some embodiments, one or more of the following may be biocompatible, allowing insertion into the blood vessel of a patient: the reinforcement fibers 28, the wall 26, the stripe 84, and the annular layer 86. In some embodiment, reinforcement fibers 28 constructed of a biocompatible material may facilitate one or more of the reinforcement fibers 28 proximate a fluid pathway extending through the catheter 14. In some embodiments, when the reinforcement fibers 28, the stripe 84, or the annular layer 85 are separated by the wall 26 from the fluid pathway, a greater range of materials may be used to construct the reinforcement fibers 28, the stripe 84, and the annular layer 85 when they are not in contact with the fluid pathway leading to the patient. In some embodiments, the first material or material of the wall 26 of the catheter 14 may include a conductive additive, which may provide a pathway for sending/receiving an electrical signal through the catheter 14. In some embodiments, the reinforcement fibers 28 may include one or more antimicrobial, anticoagulatory, or anti-fouling compounds.

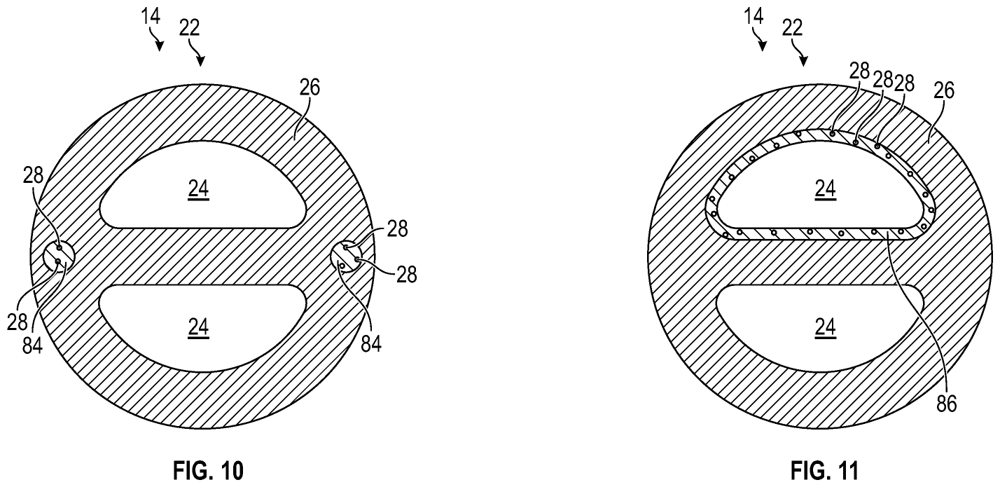

Referring now to FIG. 10, in some embodiments, the catheter 14 may include multiple of the lumens 24 extending through the shaft 22. For example, the catheter 14 may include 1 to 5 lumens 24, or another suitable number of lumens. In some embodiments, the shaft 22 may include two of the stripes 84. In some embodiments, wherein the stripes 84 may be opposite each other and/or between the lumens 24 and completely surrounded by the wall 26.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter assembly, comprising:
a catheter adapter, comprising a distal end and a proximal end; and
a catheter extending from the distal end of the catheter adapter, wherein the catheter comprises a shaft and a lumen extending through the shaft, wherein the shaft comprises a wall, a stripe or an annular layer within the wall, and a plurality of reinforcement fibers within the stripe or the annular layer, wherein each of the plurality of reinforcement fibers have a greater tensile strength than the wall, wherein each of the plurality of reinforcement fibers have a greater tensile strength than the stripe or the annular layer.

2. The catheter assembly of claim 1, wherein the wall comprises an inner surface forming the lumen and an outer surface forming an exterior of the catheter.

3. The catheter assembly of claim 2, wherein the shaft comprises the stripe, wherein the stripe is proximate the inner surface and extends partially through the wall.

4. The catheter assembly of claim 2, wherein the shaft comprises the stripe, wherein the stripe is proximate the outer surface and extends partially through the wall.

5. The catheter assembly of claim 2, wherein the shaft comprises the stripe, wherein the stripe extends through the wall from the inner surface to the outer surface.

6. The catheter assembly of claim 1, wherein the shaft comprises a plurality of stripes spaced around a circumference of the shaft, wherein the plurality of stripes comprises the stripe, wherein the wall completely surrounds each of the plurality of stripes.

7. The catheter assembly of claim 1, wherein the catheter comprises another lumen extending through the shaft, wherein the shaft comprises the stripe and another stripe, wherein the stripe and the other stripe are opposite each other between the lumen and the other lumen and completely surrounded by the wall.

8. The catheter assembly of claim 1, wherein the plurality of reinforcement fibers comprise nylon, aromatic polyamide fiber, or carbon fiber.

9. The catheter assembly of claim 1, wherein the shaft comprises the stripe, wherein the stripe is conductive.

10. The catheter assembly of claim 1, wherein a length of each of the plurality of reinforcement fibers is between 10 microns and 1,000 microns.

11. The catheter assembly of claim 1, wherein a distance of each of the plurality of reinforcement fibers from a longitudinal axis of the catheter is different, wherein a distal end of a particular one of the plurality of reinforcement fibers overlaps with a proximal end of another particular one of the plurality of reinforcement fibers.

12. The catheter assembly of claim 1, wherein the plurality of reinforcement fibers are oriented generally parallel to a longitudinal axis of the catheter.

13. The catheter assembly of claim 1, wherein the shaft comprises the annular layer within the wall, wherein the annular layer comprises an inner surface forming the lumen and an outer surface proximate the wall.

14. The catheter assembly of claim 1, wherein the shaft comprises the annular layer within the wall, wherein the wall sandwiches the annular layer.

15. A catheter assembly, comprising:
a catheter adapter, comprising a distal end and a proximal end; and
a catheter extending from the distal end of the catheter adapter, wherein the catheter comprises a shaft and a lumen extending through the shaft, wherein the shaft comprises a wall and a plurality of reinforcement fibers within the wall, wherein each of the plurality of reinforcement fibers have a greater tensile strength than the wall.

16. The catheter assembly of claim 13, wherein the plurality of reinforcement fibers comprise nylon, aromatic polyamide fiber, or carbon fiber.

17. The catheter assembly of claim 13, wherein the plurality of reinforcement fibers are conductive.

18. The catheter assembly of claim 13, wherein a length of each of the plurality of reinforcement fibers is between 10 microns and 1,000 microns.

19. The catheter assembly of claim 13, wherein each of the plurality of reinforcement fibers is encapsulated by the wall.

20. A method of manufacturing a catheter, comprising:
providing an extrusion pin within an extrusion die;
flowing a polymeric material around an exterior surface of the extrusion pin and through the extrusion die, wherein the polymeric material comprises a plurality of reinforcement fibers, wherein in response to the polymeric material flowing around the exterior surface of the extrusion pin and through the extrusion die, the plurality of reinforcement fibers are configured to orient towards a position aligned with a longitudinal axis of the catheter; and
drawing down the polymeric material out of the extrusion die, wherein in response to drawing down the polymeric material out of the extrusion die, the plurality of reinforcement fibers are configured to orient further towards the position aligned with the longitudinal axis of the catheter.
